(19) United States
(12) Patent Application Publication
(10) Pub. No.: US 2011/0039946 A1
(43) Pub. Date: Feb. 17, 2011

(54) METHOD OF IMPROVING ATOPIC DERMATOLOGICAL DISEASES, IN WHICH A COMPOSITION COMPRISING MAGNOLOL, HONOKIOL OR A COMBINATION THEREOF IS ADMINISTERED TO A PATIENT WITH ATOPIC DERMATOLOGICAL DISEASES

(75) Inventors: Deok Hoon PARK, Yongin-si (KR); Jong Sung LEE, Anyang-si (KR); Junho PARK, Yongin-si (KR); Eunsun JUNG, Suwon-si (KR); Seong Taek HONG, Incheon (KR)

Correspondence Address: CANTOR COLBURN LLP, 20 Church Street, 22nd Floor, Hartford, CT, 06103, US

(73) Assignee: BIOSPECTRUM, INC., Gunpo-si (KR)

(21) Appl. No.: 12/908,935

(22) Filed: Oct. 21, 2010

Related U.S. Application Data
(63) Continuation of application No. 11/717,265, filed on Mar. 13, 2007.

(51) Int. Cl. A61K 31/05; A61P 17/00

(52) U.S. Cl. 514/733

(57) ABSTRACT

Disclosed is a method of improving atopic dermatological diseases, in which a composition comprising magnolol, honokiol or a combination thereof is administered to a patient with atopic dermatological diseases.

This application is a continuation of U.S. patent application Ser. No. 11/717,265 filed Mar. 13, 2007, the disclosure of which is incorporated herein by reference in its entirety.

### BACKGROUND OF THE INVENTION

1. Field of the Invention

The present invention relates to a method of improving atopic dermatological diseases, in which a composition comprising magnolol, honokiol or a combination thereof is administered to a patient with atopic dermatological diseases.

2. Description of the Related Art

It has been known that atopic dermatitis is caused by complicated involvement of many various factors. Atopic dermatitis is a common chronic inflammatory dermatologic disease, which can be diagnosed with three kinds of characteristic conditions including individual or familial history, severe pruritus and eczema and may be exacerbated due to infection, mental stress, seasonal and climatic change, stimulus and allergen. The exact etiology of atopic dermatitis has not yet been clarified, but has been thought as being a hereditary disease involving immunological abnormality (Cooper, K. D. (1994) J Invest Dermatol. 102: 128-137; Bos, J. D et al., (1992) Arch Dermatol 128: 1509-1512).

In case of atopic dermatitis, type 2 T cell is mainly activated to induce the secretion of IL-4 (interleukin-4), IL-5 (interleukin-5) and IL-10 (interleukin-10). Among them, IL-4 has been reported as playing an important role in increasing IgE (immunoglobulin E) (Cooper, K. D. (1994) J Invest Dermatol. 102: 128-137; Mosmann, T. R. (1986) J Immunol 136: 2348-2357; LEE, Sung-Hun and others, (1998) Korean Journal of Dermatology 36: 95-102). In addition, it has been found that eosinophil granular proteins such as ECP (eosinophil cationic protein) are deposited on the lesion of atopic dermatitis to show the degree of inflammation, and E-selectin as the cell adhesion molecule adheres memory T-cells to endothelial cells through interaction with CLA (cutaneous lymphocyte associated antigen) to increase the infiltration into inflammation region (Venge, P. (1993) Allergy 36: 95-102; Ackerman, S. J. et al (1983) J Immunol 131: 2977-2982; Czech, W. et al (1996) Br J Dermatol 134: 17-21). The symptoms of atopic dermatitis expressed through such cell signaling pathway are mainly the cases accompanying inflammatory dermatological disorder as well as simple skin drying symptom. Therefore, the medical examination at dermatology generally includes the prescription of humectants to maintain moisture at the surface of skin, together with steroid hormones, i.e. the topical formulation of adrenal corticosteroids, to improve the inflammatory response. However, it has been reported that when topical adrenal corticosteroids are used for a long period, various dermatologic side effects including skin atrophy, vasodilation, depigmentation and striae distensae are caused (Baumann, L. et al. (1999) Dermatology in general medicine. (5th ed.), 2713-2717. Academic Press, NY). Therefore, various studies are under actively progressing in order to develop a raw material or pharmaceutical product for treating atopy with possessing anti-inflammatory effect but without causing such side effects (Robinson, N. (2001) Semin Cutan Med Surg. 20(4): 242-249).

Under such condition, the present inventor has identified that magnolol and honokiol have an excellent antibacterial effect against Staphylococcus aureus as the major causative microorganism for atopic dermatitis, exhibit a superior effect of inhibiting the production of proinflammatory cytokines induced by Staphylococcus aureus, and further, have good safety for skin of human being and effect of improving atopic dermatitis, and thus completed the present invention.

### SUMMARY OF THE INVENTION

The object of the present invention is to provide a method of improving atopic dermatological diseases, in which a composition comprising magnolol, honokiol or a combination thereof is administered to a patient with atopic dermatological diseases.

According to one aspect, the present invention provides a method of improving atopic dermatological diseases, in which a composition comprising magnolol, honokiol or a combination thereof is administered to a patient with atopic dermatological diseases.

It has been known that magnolol (5,5′-di-2-propenyl-1,1′-biphenyl-2,2′-diol) and honokiol (3,5′-di-2-propenyl-1,1′-biphenyl-2,4′-diol) have very various effects including effect as agent for insomnia, anxiolytic agent and anti-depressive agent (U.S. Unexamined Patent Publication No. US20040228934, Japanese Unexamined Patent Publication No. JP2001026537); effect as agent for inhibiting vascularization and anti-cancer agent (U.S. Unexamined Patent Publication No. US20040105903); effect as anti-plaque and anti-gingivitis agents (U.S. Pat. No. 6,500,409); effect of preventing liver and preventing and treating liver fibrosis and liver cirrhosis (Japanese Unexamined Patent Publication No. JP10045573, Korean Patent Application No. 10-2003-0053093); effect of inhibiting cholesterol absorption (Japanese Unexamined Patent Publication No. JP08003033); effect of aggregating platelet (Japanese Unexamined Patent Publication No. JP030271312), and the like.

The present inventor newly defined the effect magnolol or honokiol to improve and treat atopic dermatological diseases.

The present inventor has found that magnolol or honokiol has an excellent antibacterial effect against Staphylococcus aureus as the major causative microorganism for atopic dermatitis. Specifically, honokiol and magnolol exhibited a remarkable antibacterial effect at concentration of 6.25 μg/disk (0.39 mM) and 12.5 μg/disk (0.78 mM), respectively (Table 1). Magnolol has the minimum inhibitory concentration of 6 μg/ml and thus, exterminated Staphylococcus aureus within 10 minutes at concentration of 45 μg/ml (169.2 μM). Honokiol has the minimum inhibitory concentration of 2 μg/ml and thus, exterminated Staphylococcus aureus within 10 minutes at concentration of 20 μg/ml (75.2 μM). In addition, in treating with magnolol or honokiol the production of both IL-8 and TNF-alpha induced by Staphylococcus aureus was reduced. Further, upon preparing the topical dermatologicals comprising magnolol or honokiol and examining the efficacy thereof in patients suffering from mild or moderate atopic dermatitis, EASI values and pruritus were greatly improved. Furthermore, it has been determined from the human skin cumulative test that magnolol and honokiol have a good safety for skin.

Magnolol or honokiol included in the composition of the present invention can be either isolated from the nature (Chinese Unexamined Patent Publication Nos. CN1446785 and CN1583700, Japanese Unexamined Patent Publication No. JPO4264035) or chemically synthesized (Li C Y et al., (2003), Bioorg Med Chem. 15; 11(17):3665-71).

Magnolol or honokiol included in the composition of the present invention can be in the form of any derivative thereof. The derivatives of magnolol have been disclosed in Taiwan Unexamined Patent Publication No. TW503235, etc.

Although the composition of the present invention can contain magnolol or honokiol alone, the combination of magnolol and honokiol in the composition exhibits a better effect of improving atopic dermatologic condition. In such a case, magnolol and honokiol can be included in the ratio of 0.5˜4:1 magnolol:honokiol, preferably in the ratio of 0.5˜3:1, and more preferably in the ratio of 2:1.

The content of magnolol, honokiol or the combination thereof in the composition of the present invention can be varied, but are preferably 0.0001 wt % to 20 wt % with respect to the total weight of the composition.

The composition of the present invention is a safe substance not triggering any toxicity or stimulus in human being, and thus, can be utilized in various foods, cosmetic and medical products for the purpose to improve atopic dermatitis.

In case where the composition of the present invention is utilized in the medicinal products, it can be prepared in the form of various oral and parenteral formulations conventional in the pharmaceutical field by incorporating pharmaceutically acceptable carriers. The solid formulations, including powder, granule, tablet, capsule, etc., for oral administration can use binders, lubricants, disintegrants, excipients, solubilizers, dispersants, stabilizers, suspending agents, colorings, flavors, etc. Oral liquid formulations include suspension, enteral solution, emulsion and syrup, and can contain wetting agents, sweetners, flavors and preservatives, as well as water and liquid paraffin as the simple diluent frequently used in the pharmaceutical field. The formulations for parenteral administration include injection, ointment, suppository, inhalant, aerosol, patch, etc. The composition of the present invention can be administered in an amount of 5 to 0.01 to 10 mg/kg per each time.

Meanwhile, various pharmacologically active substances, which have been used in the prior art, can be used in combination with the composition of the present invention unless they adversely affect the pharmacological activity of the composition of the present invention. For instance, humectants such as ceramides, lipid components, steroids such as hydrocortisone, vitamin A derivatives such as retinyl palmitate and/or tocopherol, all of which have been generally used in the agent for treating atopic dermatitis in the prior art, can be used.

When the composition of the present invention is utilized in the food products, it can be formulated into various functional foods, health foods or health supplement foods including confectioneries, beverages, alcoholic drinks, fermented foods, canned foods, dairy processed foods, etc. As used in the present invention, the term “functional food” is the term identical to the food for special health use (FoSHU), and is the food having medicinal effects, which is processed so as to efficiently exhibit the biologically modulating function as well as to supply the nutrition. As used in the present invention, the term “health food” means the food having effects of actively maintaining or improving the health condition as compared to general food products; and the term “health supplement food” means the food for supplementing the health. If necessary, the terms as indicated above can be interchangeably used. The food products can be prepared in the form of tablets, capsules, powders, granules, liquids, pills, etc.

In addition, when the composition of the present invention is utilized in the cosmetic products, it can be formulated into conventional liquid or solid forms. The cosmetic products in the form of liquid or solid can include, for example, but are not limited to, skin lotions, creams, lotions, packs, soaps, facial cleansing products such as facial cleansing foam, baths, shampoos, etc. These respective formulations can contain suitable bases, auxiliaries and additives, of which the kinds and contents can be readily understood and selected by a person skilled in the relevant technical field.

In the specific working examples, the present inventor prepared the cosmetic product containing magnolol or honokiol together with glycerin, butylene glycol, caprylic capritriglyceride, squalane, cetearyl glucuside, sorbitan stearate, cetearyl alcohol, and trimethanolamine. When the magnolol cream or the honokiol cream is applied to skin, pruritus as the characteristic symptom of atopic dermatitis was also greatly improved.

### BRIEF DESCRIPTION OF DRAWINGS

FIG. 1 is the graphs showing the standard curves for antibacterial activities of magnolol (A) and honokiol (B) against Staphylococcus aureus;

FIG. 2 is the graphs showing the bactericidal curves of magnolol and honokiol against Staphylococcus aureus;

FIG. 3 is the graphs showing the inhibition by magnolol and honokiol of interleukin-8 (A) and TNF-alpha (B) production induced by Staphylococcus aureus, and showing the synergistic effect (C) between magnolol and honokiol (M: magnolol, H: honokiol, S. aureus: Staphylococcus aureus);

FIG. 4 is the graph showing the result obtained from observation of EASI as the atopic dermatitis index 2 weeks and 4 weeks after treating patients suffering from atopic dermatitis with magnolol, honokiol, or pimecrolimus cream, respectively; and

FIG. 5 is the graph showing the result obtained from observation of the degree of pruritus improvement 2 weeks and 4 weeks after treating patients suffering from atopic dermatitis with magnolol, honokiol, or pimecrolimus cream, respectively.

### DESCRIPTION OF EMBODIMENTS

Hereinafter, the present invention is specifically illustrated by the examples. However, the following examples are provided for more easy understanding of the present invention but should not be construed to limit the scope of the present invention.

### Example 1

### Antibacterial Activity of Magnolol and Honokiol Against Staphylococcus aureus

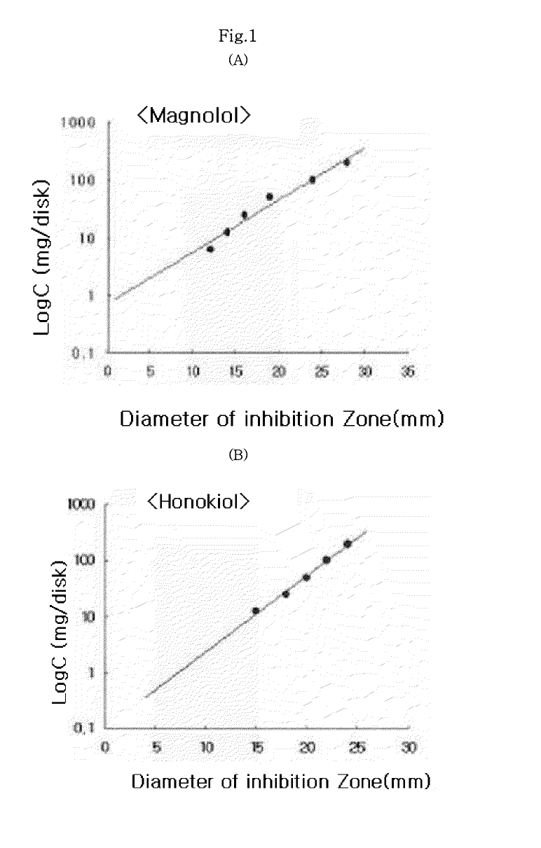

The standardized filter-paper disk agar diffusion method was used to determine the antibacterial activity of magnolol and honokiol against Staphylococcus aureus. This method was also well-known as Kirby-Bauer method, and has been widely used to determine the antibacterial activity (see, European Journal of Pharmacology, Junho Park et al, 2004, 496(1-3), 189-195). Briefly, Staphylococcus aureus cells were incubated with brain heart infusion (BHI) broth at 37° C. under anaerobic conditions until they reach the stationary phase. Approximately 1×10⁶ bacteria cells were mixed with 7 ml of BHI agar medium containing 0.8% phytoagar and then, poured on the agar plate containing 1.5% phytoagar. The filter-paper discs (10 mm diameter) were treated with magnolol and honokiol at respective test concentrations, and then placed on the BHI agar plate inoculated with the test bacteria. The plate was incubated at 37° C. for 24 hours under anaerobic conditions, and then the antibacterial activity was determined by measuring the size of clear zone (growth inhibition halo) around the disk. The size of clear zone has the functional relation with the logarithmic value of magnolol or honokiol concentration, from which the standard calibration curve could be derived (FIG. 1). The unknown concentration of the test sample can be determined from this standard calibration curve. As presented in Table 1, both of magnolol and honokiol exhibited a significant antibacterial activity against Staphylococcus aureus. The antibacterial activity was remarkable at 6.25 μg/disk (0.39 mM) for honokiol and 12.5 μg/disk (0.78 mM) for magnolol (Table 1).

TABLE 1
Diameter of clear zone (mm)

| | Microorganism | | | |
|---|---|---|---|---|
| | S. aureus | | | |
| Compounds | 6.25 | 12.5 | 25 | 50^{a} |
| Honokiol | 12 ± 0.4^{c} | 14 ± 0.2^{c} | 17 ± 0.8^{c} | 22 ± 1.2^{c} |
| Magnolol | —^{b} | 11 ± 0.4^{c} | 15 ± 0.2^{c} | 18 ± 0.2^{c} |
| Erythromycin | 35 ± 2.2^{c} | 42 ± 3.8^{c} | 45 ± 2.5^{c} | 48 ± 5^{c} |

^{a}Amt of compounds (mg/disk)
^{b}No antibacterial activity
^{c}P < 0.05, diameter of filter disk is 10 mm

### Example 2

### Minimum Inhibitory Concentration (MIC) of Magnolol and Honokiol

The test was conducted to determine the Minimum Inhibitory Concentrations (MIC) of magnolol and honokiol. Staphylococcus aureus incubated for 24 hours was introduced into 3 ml of brain heart infusion broth, and at the same time, magnolol and honokiol were respectively added thereto, and then incubated for 24 hours at 37° C. under anaerobic conditions. To determine MICs of honokiol and magnolol the two-fold serial dilution method was used. As used herein, MIC is defined as the minimum concentration at which the growth of microorganism is inhibited. As presented in Table 2, it could be confirmed that MICs of magnolol and honokiol for Staphylococcus aureus were 6 μg/ml and 2 μg/ml, respectively. That is, it can be seen that the antibacterial activity of honokiol is higher than that of magnolol.

TABLE 2

MIC of agents (μg/ml)

| Microorganism | Honokiol | Magnolol |
|---|---|---|
| S. aureus | 2 | 6 |

### Example 3

### Determination of the Minimum Bacteriocidal Concentration (MBC) of Magnolol and Honokiol for Staphylococcus aureus

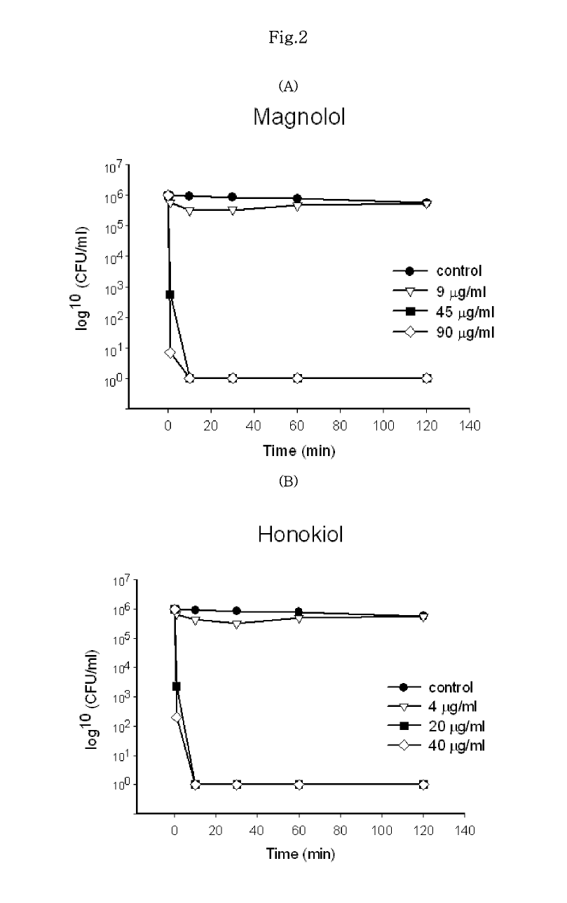

The inhibition of bacterial growth with a certain substance is the result obtained by inhibiting the proliferation of respective cells or killing the cells. The growth of bacteria can be again initiated by removing such substance in case where the substance exhibits the inhibition of proliferation (bacteriostatic activity), but could not be restarted in case of the killing of bacteria. The following experiment was conducted to determine the Minimum Bacteriocidal Concentration (MBC). First, the same procedures as the determination of MIC were conducted in the liquid culture medium, and then a portion of the culture medium in the test tube in which no proliferation was shown was taken, diluted to remove magnolol and honokiol therefrom, and then incubated on the solid culture medium and observed for the colony formation after 24 hours. At this time, MBC means the minimum value of magnolol or honokiol concentration in the original culture solution in the plate medium on which no colony was produced. To determine MBC of magnolol and honokiol Staphylococcus aureus was used, and magnolol or honokiol was applied at the concentration one, five and 10 times as high as MIC concentration thereof to determine the kinetics of bacterial killing. The survival rate of microbes was determined within 2 hours. As shown in FIG. 2, it can be seen that the cell killing rate is dependent on the concentration of magnolol or honokiol as applied, and observed that Staphylococcus aureus was completely exterminated at concentration of 20 μg/ml (75.2 μM) for honokiol and of 45 μg/ml (169.2 μM) for magnolol within 10 minutes (FIG. 2).

### Example 4

### Evaluation of the Effect of Magnolol and Honokiol of Inhibiting the Production of Proinflammatory Cytokines Induced by Staphylococcus aureus

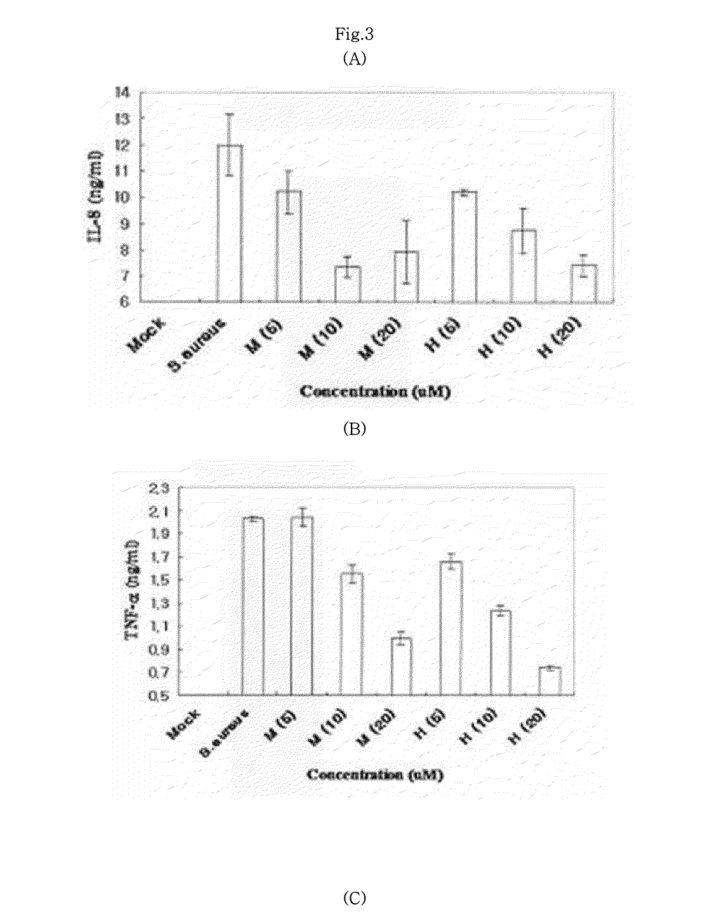

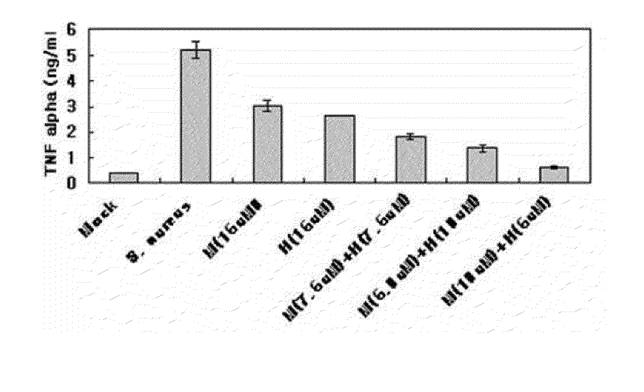

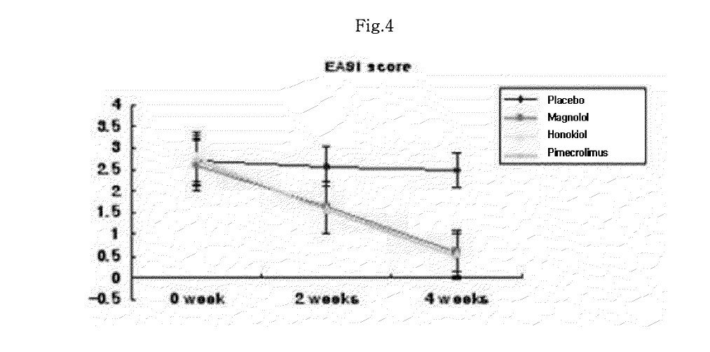

In general, the inflammatory response generated by bacteria or bacterial components is mediated by proinflammatory cytokines. To determine whether magnolol or honokiol can inhibit the inflammatory response generated by Staphylococcus aureus ELISA for interleukin-8 and TNF-alpha was conducted. For this purpose, THP-1 cell line as human monocyte cell line was used. 1×10⁶ THP-1 cells were incubated for 14 hours under serum-free conditions, and then treated with heat-inactivated Staphylococcus aureus 100 μg/ml alone or simultaneously with magnolol and honokiol, respectively, which was then incubated for 48 hours. The amounts of interleukin-8 and TNF-alpha secreted into cell culture medium were measured by means of ELISA kit (Genzyme, Minneapolis, Minn.). As shown in FIG. 3, it could be observed that the production of interleukin-8 and TNF-alpha induced by Staphylococcus aureus was reduced by both magnolol and honokiol. Generally, honokiol exhibits somewhat stronger cytokine-inhibiting efficacy at tested concentrations as compared to magnolol.

In addition, the experiment was conducted to identify the synergistic anti-inflammatory effect when magnolol and honokiol are combined in a certain ratio. The efficacy to inhibit the production of TNF-alpha was examined in cases where the combined ratio of magnolol and honokiol is 1 (7.5 μM):1 (7.5 μM), 1 (5 μM):2 (10 μM), and 2(10 μM):1 (5 μM) or in case of treating with 15 μM of magnolol alone or 15 μM of honokiol alone. As shown in FIG. 3c, the result to most effectively inhibit the production of TNF-alpha induced by Staphylococcus aureus could be obtained by treating with magnolol and honokiol combined in a certain ratio, i.e. in a ratio of 2:1, as compared to treatment with magnolol or honokiol alone. Such result suggests that the combination of magnolol and honokiol can produce a synergistic interaction, and further demonstrates that the highest synergistic effect can be obtained when a certain combined ratio of magnolol and honokiol is 2:1.

### Example 5

### Efficacy to Treat Atopic Dermatitis of the Skin Topical Preparation Containing Magnolol and Honokiol

The cosmetic compositions for atopy containing magnolol, honokiol and pimecrolimus, respectively were prepared with the contents of components as listed in the following Table 3, according to the conventional method. In the following Table 3, the control group is for the skin topical preparation with no antibacterial and anti-inflammatory substance, and the test groups 1, 2 and 3 are for the cosmetic compositions for skin topical use to treat atopy containing magnolol, honokiol and pimecrolimus, respectively.

To prepare the skin topical preparation purified water, glycerin and butylene glycol were mixed and dissolved at 70° C. (aqueous part), and the remaining components except for the three components and trimethanolamine were dissolved at 70° C. (oil part). The oil part was added to the aqueous part and stirred with a homomixer (from Tokushu Kika Kogyo, Co., Ltd. In Japan) to induce the first emulsification to which trimethanolamine was finally added. After removing bubbles formed in the mixed solution, the resulting solution was cooled to room temperature to prepare the desired topical dermatological preparation.

TABLE 3

| | Content (%) | | | |
|---|---|---|---|---|
| | Control | Test | Test | Test |
| Components | group | group 1 | group 2 | group 3 |
| Purified water | 72 | 72 | 72 | 72 |
| Glycerin | 8.0 | 8.0 | 8.0 | 8.0 |
| Butylene glycol | 4.0 | 4.0 | 4.0 | 4.0 |
| Magnolol | 0 | 1.0 | 0 | 0 |
| Honokiol | 0 | 0 | 1.0 | 0 |
| Pimecrolimus | 0 | 0 | 0 | 1.0 |
| Caprylic capritriglyceride | 8.0 | 8.0 | 8.0 | 8.0 |
| Squalane | 5.0 | 5.0 | 5.0 | 5.0 |
| Cetearyl glucuside | 1.5 | 1.5 | 1.5 | 1.5 |
| Sorbitan stearate | 0.4 | 0.4 | 0.4 | 0.4 |
| Cetearyl alcohol | 1.0 | 1.0 | 1.0 | 1.0 |
| Trimethanolamine | 0.1 | 0.1 | 0.1 | 0.1 |
| Total weight | 100 | 100 | 100 | 100 |

The clinical evaluation was conducted for the skin lotions of the control group and test groups 1-3 to determine the clinical effect of treating or alleviating atopy of the skin topical cosmetic compositions containing magnolol and honokiol. The clinical evaluation method is as follows.

### Study Method

1. Selection of the Subjects for the Study

30 children patients having the age from 6 to less than 15 who suffer from mild or moderate atopic dermatitis were used as the subject for the study.
2. Inclusion Criterion of the Subjects
2-1. Inclusion Criterion

(1) Criterion to diagnose atopic dermatitis: In case where 3 or more of major symptoms and 3 or more of accidents are present on the basis of the diagnosis criterion made by Hanifin and Rajka in 1980, which has been commonly and widely used in worldwide at present, they were diagnosed as atopic dermatitis.

(2) Male or female volunteers having the age from 6 to less than 15 and suffering from mild or moderate atopic dermatitis

(3) Patients who do not fall under the exclusion criterion of subjects

(4) Volunteers spontaneously signed on the written informed consent after hearing full explanation of the purpose and details of the test before starting the test

(5) Volunteers who are capable of monitoring during the test period
2-2. Exclusion Criterion

(1) Patients with severe atopic dermatitis

(2) Patients who are under treatment with immunosuppressive agents, and immunomodulating agents for severe atopic dermatitis

(3) Cases determined as being difficult to conduct the study on judgment by a person in charge of the study, in addition to the above conditions
3. Clinical Test Method

The test was conducted in the manner of the prospective open study as follows by applying the magnolol, honokiol or pimecrolimus creams to patients suffering from atopic dermatitis. The individual application or administration of topical or systemic steroidal preparations was prohibited except for the anti-histaminic preparations supplied for severe pruritus.
4. Method for Evaluating the Effect
4-1. Clinical Evaluation of Atopic Dermatitis

1) The symptoms and extent of atopic dermatitis were objectively determined using EASI (Eczema Area Severity Index) as follows, before treatment and 2 and 4 weeks after treatment.

| Body regions | EASI Score |
|---|---|
| Head/Neck (H) | (E + I + Ex + L) × area × 0.1 |
| Upper Limbs (UL) | (E + I + Ex + L) × area × 0.2 |
| Trunk (T) | (E + I + Ex + L) × area × 0.3 |
| Lower Limbs (LL) | (E + I + Ex + L) × area × 0.4 |
| EASI | Sum of the above 4 body region scores |

* E = Erythema, I = induration/population, Ex = excoriation, L = lichenification (0 = none, 1 = mild, 2 = moderate, 3 = severe)
* Area: 0 = no eruption, 1 = <10%, 2 = <10-29%, 3 = <30-49%, 4 = <50-69%, 5 = <70-89%, 6 = >90-100%

2) Evaluation of pruritus

Visual analog scale Pruritus 0 1 2 3 4 5 6 7 8 9 10
4-2. Statistical Analysis

EASI score and pruritus score were compared and evaluated by applying Kruskall-Wallis one-way ANOVA method as the basic statistical method before treatment and 2 and 4 weeks after treatment. When the p value is below 0.05 as the effective deviation obtained by statistically verifying the improvement and difference after treatment, the result was judged as being significant.

### Result

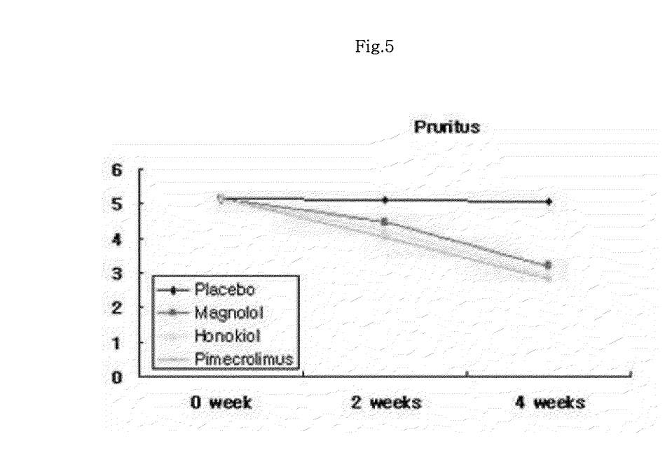

32 subject patients were allowed to use the cream preparations containing magnolol, honokiol and pimecrolimus, respectively, and 2 of them dropped out due to retrogression of clinical symptoms and an increase of pruritus in using the preparation, during the study period. The following results are those derived by analyzing the results for 30 patients (11 male patients and 19 female patients, average age 7.9 years of old). As can be seen from the result depicted in FIG. 5, it could be noted that when the magnolol creams and the honokiol creams are applied, EASI as the index for atopic dermatitis is began to decrease after 2 weeks and greatly decreased after 4 weeks. The pimecrolimus creams used as the (+) control group also exhibit a tendency similar to magnolol and honokiol (FIG. 5). Furthermore, it could be observed that pruritus accompanied by atopic dermatitis is significantly improved by magnolol and honokiol (FIG. 6).

From FIGS. 5 and 6, it was confirmed that the skin topical preparations containing magnolol and honokiol according to the present invention have a good effect of treating or alleviating atopy. Particularly, it can be seen that their effect is similar to the effect of pimecrolimus, which has been generally used in the prior art.

### Example 6

### Test to Ascertain the Safety of Magnolol and Honokiol for Human Skin

To ascertain whether magnolol and honokiol, which were confirmed as having good effect of improving atopy, are also safe for human skin the test to verify the skin safety was conducted for the test groups 1 to 3 as listed in Example 5. The test was practiced according to the human skin cumulative test.

Each skin topical preparation prepared in Example 5 was used for 30 healthy adult subjects by applying the 24-hours cumulative patch on the upper forearm region every other day in a total of 9 times, to determine whether magnolol and honokiol irritate the skin.

The patch method utilized Finn chamber (Epitest Ltd., Finland). 15 μl of each of the skin topical preparations was added dropwise to the chamber and then the patch was applied. The degree of response shown on the skin each time was scored by using the following equation, and the result obtained therefrom is listed in the following Table 4.

Average response degree=[[Response index×Response degree/Total number of subjects×Highest score (score 4)]×100]÷Times of examination (9 times)

Herein, the response degrees ±, + and ++ give the scores 1, 2 and 4, respectively, and the composition having the average response degree less than 3 is judged as being safe.

TABLE 4

| | Number of subjects showing the response | | | | | | | | | | | | | | | | | | | | | | | | | | | Average |
|---|---|---|---|---|---|---|---|---|---|---|---|---|---|---|---|---|---|---|---|---|---|---|---|---|---|---|---|---|
| | One week | | | | | | | | | Two week | | | | | | | | | Three week | | | | | | | | | re- |
| Test | 1st | | | 2nd | | | 3rd | | | 4th | | | 5th | | | 6th | | | 7th | | | 8th | | | 9th | | | sponse |
| substance | ± | + | ++ | ± | + | ++ | ± | + | ++ | ± | + | ++ | ± | + | ++ | ± | + | ++ | ± | + | ++ | ± | + | ++ | ± | + | ++ | degree |
| Control | 2 | — | — | — | — | — | — | — | — | — | — | — | — | — | — | — | — | — | — | — | — | — | — | — | — | — | — | 0.18 |
| group | | | | | | | | | | | | | | | | | | | | | | | | | | | | |
| Test | 2 | — | — | 0 | — | — | — | — | — | — | — | — | — | — | — | — | — | — | — | — | — | — | — | — | — | — | — | 0.18 |
| group 1 | | | | | | | | | | | | | | | | | | | | | | | | | | | | |
| Test | 3 | — | — | 1 | — | — | — | — | — | — | — | — | — | — | — | — | — | — | — | — | — | — | — | — | — | — | — | 0.37 |
| group 2 | | | | | | | | | | | | | | | | | | | | | | | | | | | | |
| Test | 3 | — | — | 1 | — | — | — | — | — | — | — | — | — | — | — | — | — | — | — | — | — | — | — | — | — | — | — | 0.37 |
| group 3 | | | | | | | | | | | | | | | | | | | | | | | | | | | | |

| | | | | | | | | | |
|---|---|---|---|---|---|---|---|---|---|
| Number of | 30 | 30 | 30 | 30 | 30 | 30 | 30 | 30 | 30 |
| subjects | | | | | | | | | |

In case of the test group 1, the number of subjects falling under ±, + and ++ were 2, 0 and 0, respectively, and the remaining subjects did not exhibit any response. When calculating the average response degree according to the above equation, the average response degrees of the test groups 1 to 3 were 0.18, 0.37 and 0.37, respectively, all of which are below 3. Therefore, the composition of the present invention could be determined as being the safe composition.

As described in Table 4, the skin topical preparations containing magnolol (Test group 1) and honokiol (Test group 2) did not exhibit a remarkable cumulative irritation aspect as in the control group or the skin topical preparation containing pimecrolimus (Test group 3). Therefore, they could be determined as being a safe substance.

As reviewed above, the composition comprising magnolol, honokiol or the combination thereof according to the present invention is a safe substance not causing any toxicity or irritation in the human body, and therefore, can be usefully used in the cosmetic or food product for improving atopic dermatological diseases.

What is claimed is:

1. A method of improving atopic dermatological diseases, in which a composition comprising magnolol, honokiol or a combination thereof is administered to a patient with atopic dermatological diseases.

2. The method according to claim 1, wherein the composition comprises the combination of magnolol and honokiol.

3. The method according to claim 2, wherein the composition comprises magnolol and honokiol in a molar ratio of 0.5-4:1.

4. The method according to claim 1, wherein the composition is formulated in a skin topical preparation.
